(19) United States
(12) Patent Application Publication
(10) Pub. No.: US 2002/0173016 A1
(43) Pub. Date: Nov. 21, 2002

(54) High-throughput nucleic acid polymerase devices and methods for their use

(76) Inventors: Helmut Wurst, Cupertino, CA (US); Ningyue Zhao, Milpitas, CA (US)

Correspondence Address: Bret E. Field, BOZICEVIC, FIELD & FRANCIS LLP, 200 Middlefield Road, Suite 200, Menlo Park, CA, 94025, US

(21) Appl. No.: 09/820,085

(22) Filed: Mar. 27, 2001

(51) Int. Cl. C12N 11/00; C12N 11/16; C12N 9/22

(52) U.S. Cl. 435/174; 435/199

(57) ABSTRACT

High throughput lyophilized polymerase devices and methods for their use in the production of nucleic acids using template dependent polymerase reactions are provided. The subject devices are typically made up of a multi-well substrate that includes in a least one well a lyophilized nucleic acid polymerase composition. The subject nucleic acid polymerase compositions include at least one polymerase and a carbohydrate stabilizing composition that is made up of at least one low molecular weight sugar and a starch. In many embodiments, the compositions also include buffer components and nucleotides, as well as a temperature dependent polymerase inhibitor, e.g., a polymerase specific antibody. Also provided are kits that include the subject devices.

### INTRODUCTION

1. Technical Field

The field of this invention is template dependent nucleic acid synthesis.

2. Background of the Invention

Template dependent or template driven nucleic acid synthesis is a protocol that finds use in a multitude of prominent biotechnology applications, including the polymerase chain reaction (PCR); cDNA synthesis, and the like. In template driven nucleic acid synthesis, one or more DNA polymerases are used in the presence of a template nucleic acid, a primer nucleic acid, magnesium ions and deoxynucleotide triphosphates (dNTPs), as well as appropriate buffer components, to synthesize deoxyribonucleic acid (DNA).

In traditional template driven nucleic acid synthesis protocols, all of the various reagents are added in appropriate amounts to produce a reaction mixture. Because of the multiplicity of reagents that are employed and the requirements for precise measuring, such traditional protocols are time consuming, inefficient and prone to contamination.

In order to overcome the above problems experienced with traditional protocols, certain “ready-to-use” compositions in which all of the necessary reagents are precombined into a single aqueous composition have been developed for sale to the academic and research markets. Such compositions, typically referred to as “master-mixes” are available from a number of different commercial vendors. However, because such compositions are aqueous compositions, they require special storing and shipping procedures, e.g., storage at sub freezing temperatures, which is a disadvantage.

As such, there is an interest in the development of room temperature storage stable alternatives to the aqueous master mixes currently being marketed. One room temperature stable alternative that is currently marketed is the “Ready to go PCR Beads” product marketed by Amersham Pharmacia Biotech. While this product overcomes certain of the problems experienced with aqueous master-mixes, e.g., it can be stored at room temperature, it must be stored with a desiccant and handled prior to use. Furthermore, this product is deigned for use in PCR with Taq polymerase only, and “long distance” formulations and/or “hot start” formulations are not available in this format. This technology is further described in U.S. Pat. No. 5,593,824.

As such, there is continued interest in the development of additional dry, storage stable polymerase compositions for use in template driven nucleic acid synthesis protocols. Of particular interest would be the development of such compositions that require minimal handling and are readily adapted for use in high-throughput applications.

3. Relevant Literature

Of interest is U.S. Pat. No.5,593,824. See also, Klaster et al., “Stabilized, Freeze-Dried PCR Mix for Detection of Mycobacteria,” J. Clin. Microbiol. (June 1998) 36:1798-1800; “Ready to Go PCR Beads” Amersham Pharmacia Biotech online product catalog description, available on the world wide web at least as early as Feb. 12, 2001.

### SUMMARY OF THE INVENTION

High throughput devices and methods for their use in the production of nucleic acids using template-dependent polymerase reactions are provided. The subject devices are typically made up of a multi-well substrate that includes in a least one well a lyophilized nucleic acid polymerase composition. The subject nucleic acid polymerase compositions include at least one polymerase and a carbohydrate stabilizing composition that is made up of at least one low molecular weight sugar and a natural carbohydrate polymer, e.g., naturally occurring starch. In many embodiments, the compositions also include buffer components and nucleotides. In many embodiments, the subject wells are heat sealed under atmospheric pressure to minimize exchange of air in the wells and thus, to minimize uptake of moisture from the air. This configuration allows storage over prolonged periods of time in the absence of a dessicant. Also provided are kits that include the subject devices.

### DESCRIPTION OF THE SPECIFIC EMBODIMENTS

High throughput devices and methods for their use in the production of nucleic acids using template-dependent polymerase reactions are provided. The subject devices are typically made up of a multi-well substrate that includes in a least one well a lyophilized nucleic acid polymerase composition. The subject nucleic acid polymerase compositions include at least one polymerase and a carbohydrate stabilizing composition that is made up of at least one low molecular weight sugar and a starch. In many embodiments, the compositions also include buffer components and nucleotides. Also provided are kits that include the subject devices. In further describing the subject invention, the subject high throughput devices will be described first, followed by a discussion of the multi-well formats thereof and a review of the subject methods of template dependent nucleic acid synthesis. Finally, the subject kits will be described in greater detail.

Before the subject invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

### HIGH THROUGHPUT DEVICES

As summarized above, the subject invention is directed to high-throughput devices that include a lyophilized nucleic acid polymerase composition. The subject devices provide for a high throughput storage stable format of a lyophilized nucleic acid polymerase composition. As such, the invention is directed to room temperature storage stable high throughput lyophilized nucleic acid compositions. By storage stable is meant that the subject devices may be maintained at ambient conditions, e.g., ambient temperature and humidity, for at least about 1 month, usually at least about 6 months, and more usually at least about 1 year, without adverse consequences to the lyophilized nucleic acid polymerase composition present in the device. Typically, the compositions are storage stable at temperatures ranging from about 15 to 30, usually from about 18 to 25° C. Long term storage of the subject devices within these temperature and humidity ranges does not substantially adversely effect the components of the lyophilized composition, such that the lyophilized composition when reconstituted with water can still be used for template driven nucleic acid synthesis applications. As such, the subject devices are properly characterized as “room-temperature stable.” By “room-temperature stable” is meant that the devices can be stored at 22° C. for greater than six months with less than 20% loss of polymerase activity as compared to the activity measured after the polymerase composition is first dried.

The high throughput devices of the present invention are typically made up of multi-well substrates, or analogous structures that include a plurality of liquid containment means, where the devices include in at least one or more of their wells a lyophilized nucleic acid polymerase composition. The subject substrates typically have a plurality of reaction chambers or wells. By plurality is meant at least 2, usually at least 6, more usually at least 24, and most usually at least 96, e.g., 96, 384 etc., where the number of different reaction chambers of the device may be as high as 450 or higher, e.g., 1000 well plates, 1500 well plates, 2000 well plates, etc. In many embodiments, of particular interest are 96 and 384 well devices. A representative substrate is a PCR plate, where representative multiwell PCR plates are currently commercially available, e.g., from ABgene North America, 98 College Avenue, Rochester, N.Y. 14607. The overall size and configuration of the device will be one that provides for simple, manual handling, where the device may be disc shaped, slide shaped, and the like, where slide shaped (i.e. having a substantially rectangular cross-sectional shape, such as found in a microscope slide or a credit card) is preferred. The length of the device typically ranges from 10 to 1000 mm, usually from 20 to 200 mm and more usually from 100 to 150 mm, the height of the device ranges from 1 to 50 mm, usually from 5 to 30 mm and more usually from 10 to 25 mm and the width of the device ranges from 10 to 1000 mm, usually from 20 to 200 mm and more usually from 50 to 150 mm.

Each reaction chamber of the device is a container having an open top and at least one wall configured in a manner sufficient to form a fluid container, where the number of distinct walls depends on the cross-sectional shape of the container, e.g. 1 wall for a container having a circular cross-sectional shape and 4 walls for a container having a square cross-sectional shape. Typically, the structure of the well is conical, such that the wall(s) gradually merge to a point at the bottom of the well. However, other configurations are also encompassed, such as wells that have a distinct bottom surface, e.g., a planar bottom surface. The reaction chamber may have a variety of cross-sectional shapes, including circular, triangular, rectangular, square, pentagon, hexagon, etc., including irregular, and in many embodiments has a circular, rectangular or square cross-sectional shape. Therefore, the number of distinct walls of the well is at least one, and can be 2, 3, 4, 5, 6 or more, depending on the cross-sectional shape.

The volume of fluid capable of being contained in the reaction chambers or wells generally ranges from about 0.0001 to 10 ml, usually from about 0.001 to 1 ml and more usually from about 0.005 to 0.1 ml. The height of the walls will generally be uniform and will be sufficient to form a reaction chamber that is capable of holding a desired amount of fluid, e.g. reaction medium. As such, the height of each wall of the reaction chamber will be at least about 0.1 mm, usually at least about 1 mm and more usually at least about 10 mm and may be as high as 20 mm or higher, but will usually be no higher than about 50 mm, and more usually no higher than about 100 mm.

The material from which the multi-well substrates are fabricated will generally be a rigid material capable of providing a physical support and structure to the device, where suitable materials include: glass (glasses or silicon), plastics, e.g., polytetrafluoroethylene, polyvinylidenedifluoride, polypropylene, polystyrene, polycarbonate, combinations or modifications thereof (particularly heat stable materials), and the like, metals, e.g. aluminum, gold, titanium, or alloy and the like, where in many embodiments the material will preferably be transparent, at least to certain light wavelengths. The walls and the bottom surface of the reaction chambers making up the subject devices may be fabricated from the same or different materials.

The multi-well substrates may be fabricated using any convenient methodology, where a variety of such methodologies are known to those of skill in the art. Depending on the nature of the material from which the device is fabricated, fabrication may involve injection molding, micro machining or other suitable fabrication techniques. The device may be prepared as a single unit, or the bottom portion of the device may be fabricated separately from the walls, where the device is then assembled by placing the walls on the surface of the plate in a stable relationship.

As mentioned above, present in at least one well of the multi-well substrate is a lyophilized nucleic acid polymerase composition. A feature of the subject lyophilized compositions is the presence of at least one nucleic acid polymerase and a carbohydrate stabilizing component, where these and other components are present in a freeze-dried composition. In the broadest sense, any nucleic acid polymerase may be present in the subject compositions, where the polymerase may be a DNA polymerase, an RNA polymerase, a reverse transcriptase, etc. In many embodiments, the polymerase is a DNA-dependent DNA polymerase.

Of particular interest in many embodiments are compositions where at least one polymerase is a thermostable DNA polymerase. Thermostable DNA polymerases of interest include: Thermus aquaticus DNA polymerase; Thermus thermophilus DNA polymerase; Thermococcus litoralis DNA polymerase; Thermotoga maritima DNA polymerase; Pyrococcus furiosus DNA polymerase; as well as mutants, including point and deletion mutants, thereof; and the like.

In many embodiments, the thermostable DNA polymerase of the enzyme compositions of the subject invention is characterized by having substantial polymerase activity, specifically DNA dependent DNA polymerase activity, but substantially no nuclease activity, and more particularly substantially no exonuclease activity. Since the enzyme has substantial polymerase activity, it is capable of catalyzing the synthesis of DNA from deoxynucleotide triphosphates and a DNA primer using a DNA strand as a template. Since the subject polymerase lacks nuclease activity, it is incapable of catalyzing the hydrolysis of the phosphodiester bonds of DNA polymers. By substantial polymerase activity is meant that the polymerase activity of the enzyme is at least about 80,000 units/mg protein. (Polymerase activity is determined by incubating 5 μl of diluted enzyme fractions with 5 μg of activated calf thymus DNA (Worthington, Freehold, N.J.) in a buffer containing 25 mM TAPS-KOH pH 9.3, 50 mM KCL, 5 mM MgCl₂, 1.4 mM β-mercaptoethanol, 200 μM each dNTP and α-³²P dCTP (30-80 cpm/pmol) for 10 min at 72° C. in a total volume of 50 μl. The reaction is terminated by addition of 10 μl of 60 mM EDTA, and the products are precipitated by the addition of 60 μl of 20% trichloroacetic acid and incubation on ice for 15 min. The acid-insoluble product is then separated from the acid soluble nucleotides by filtration through GF/C filters. One unit represents conversion of 10 nmol of nucleotides in 30 min at 72° C.) By thermostable is meant that the enzyme maintains its polymerase activity at temperatures at least in excess of 55° C. and up to about 72° C. or higher. The thermostable polymerase is further characterized by having a higher Mg²⁺ optimum as compared to wild type Taq polymerase (Barnes, W. M., Gene (1992) 112:29-35.

Generally, the thermostable polymerase has a molecular weight that is less than the molecular weight of naturally occurring or wild type Thermus aquaticus polymerase. The molecular weight of polymerases finding use in the subject compositions typically ranges from about 60 to 70 kDal, usually from about 62 to 68 kDal, and more usually from about 64 to 68 kDal, as measured by SDS-PAGE electrophoresis. The thermostable polymerase typically has an amino acid sequence in which the C-terminal portion is substantially identical to the carboxy domain of the naturally occurring Thermus aquaticus DNA polymerase as reported in Lawyer et al., J. Biol. Chem (1989) 264:6427 and having a Genbank accession no J04639, particularly amino acid residues 289 to 832 of the naturally occurring Thermus aquaticus DNA polymerase. By substantially identical or the same is meant that the C-terminal portion of subject enzyme, which is from about 530 to 550 amino acids in length, usually from about 540 to 550 amino acids in length and more usually 540 to 545 amino acids in length, where in many instances it is 543 amino acids in length, has an amino acid sequence that has a sequence identity of at least about 90%, usually at least about 95% and more usually at least about 99%, with residues 289 to 832 of the amino acid sequence of naturally occurring Thermus aquaticus polymerase, as measured using the BLAST algorithm, as described in Altschul et al., (1990) J. Mol. Biol. 215: 403-410 (using the published default settings). In many embodiments of the subject invention, the C-terminal 543 amino acid residues, e.g. 10 to 553, 17 to 560, etc, depending on the particular embodiment of the invention, of the polymerase are identical to residues 289 to 832 of wild type Thermus aquaticus polymerase. Where the amino acid sequence of the C-terminal domain of the polymerase does differ from residues 289 to 832 of the naturally occurring sequence, the difference is not one that significantly provides for a significantly reduced polymerase activity or specificity as compared that observed for the wild type enzyme, where any reduced polymerase activity will not exceed a 4-fold reduction, and usually will not exceed a 2 to 3 fold reduction.

Adjacent to the C-terminal domain described above is the N-terminal region of the enzyme. The N-terminal region at least comprises a sequence of nine amino acid residues that has less than 50% but at least 40% amino acid sequence identity with residues 280 to 288 of naturally occurring Thermus aquaticus polymerase, as measured using the BLAST algorithm described above, where the number of amino acid residues in the N-terminal domain that are identical with residues 280 to 288 is usually four.

Generally, the sequence of this nine residue domain is:

MRGHEX₁GLX₂

wherein X₁ and X₂ are hydrophilic residues, more specifically, polar uncharged hydrophilic residues. X₁ is usually either threonine or serine, and in many preferred embodiments is serine. X₂ is usually either asparagine or glutamine, and in many preferred embodiments is glutamine.

A preferred thermostable enzyme is further described in U.S. Pat. No. 6,130,045, the disclosure of which is herein incorporated by reference.

The subject polymerase may be used as the sole polymerase in the lyophilized composition, or combined with one or more additional polymerases as desired, e.g., for the production of long PCR products. Where the subject polymerase is used as the sole polymerase in the lyophilized composition, it is present in amounts sufficient to provide for an aqueous reaction mixture upon combination with a suitable amount of water, e.g., 50 μl, that comprises from about 0.1 U/μl to 1 U/μl of the subject polymerase, usually from about 0.2 to 0.5 U/μl of the subject polymerase, where “U” corresponds to incorporation of 10 nmol dNTP into acid-insoluble material in 30 min at 74° C.

Where the subject polymerase is combined with an additional polymerase, the additional polymerase will generally be a Family B polymerase, where the such polymerase are described in Braithwaite & Ito, Nucleic Acids Res. (1993) 21:787-802. Family B polymerases of interest include Thermococcus litoralis DNA polymerase (Vent) as described in Perler et al., Proc. Natl. Acad. Sci. USA (1992) 89:5577; Pyrococcus species GB-D (Deep Vent); Pyrococcus furiosus DNA polymerase (Pfu) as described in Lundberg et al., Gene (1991) 108:1-6, Pyrococcus woesei (Pwo) and the like. Where the subject polymerase is combined with an additional Family B polymerase, the subject polymerase will be present in an activity greater than the Family B polymerase, where the difference in activity will usually be at least 10-fold, and more usually at least about 100-fold. Accordingly, the amount of this proof reading polymerase present in the lyophilized composition is sufficient to provide for an aqueous reaction mixture upon combination with 50 μl of water that comprises from about 0.1 U/μl to 1 U/μl of the nuclease deficient polymerase, usually from about 0.2 to 0.5 U/μl of the nuclease deficient polymerase and from about 0.01 mU/μl to 10 mU/μl, usually from about 0.05 to 1 mU/μl and more usually from about 0.1 to 0.5 mU/μl of the proof reading enzyme, where U corresponds to incorporation of 10 nmol dNTP into acid-insoluble material in 30 min at 74° C. In a preferred embodiment, the subject polymerase will be combined with Deep Vent polymerase, where the ratio of activity of the subject polymerase to Deep Vent will range from 50 to 10,000, more usually from 500 to 1000.

In terms of % by weight of the dry lyophilized compositions for a 50 μl PCR reaction, the total amount of the polymerase component (i.e. the one or more polymerases taken together), typically ranges from about 2.0 to 0.002, usually from about 0.2 to 0.01% w/w. Where the polymerase component of the lyophilized composition is made up of a nuclease deficient polymerase and a proof reading polymerase, the amount of nuclease deficient polymerase is typically present in amounts ranging from about 2 to 0.002%, usually from about 0.2 to 0.01% w/w of the dry composition while the amount of proof reading polymerase is typically present in amounts ranging from about 0.003 to 0.000002, usually from about 0.0003 to 0.00001% w/w of the dry composition.

In addition to the subject polymerase component, the lyophilized compositions of the present invention also include a carbohydrate stabilizing component. The amount of carbohydrate stabilizing component present in the composition is sufficient to provide for the desired stabilizing effect, where the carbohydrate component typically makes up about 20 to 90%, usually from about 50 to 80% and more usually from about 60 to 75% (w/w) of the lyophilized composition.

The carbohydrate stabilizing component is made up of at least one low molecular weight sugar and a starch. In many embodiments, the components of the carbohydrate stabilizing component are naturally occurring, i.e., non-synthetic, low molecular weight sugars and starches. By low molecular weight sugar is meant a saccharide that does not exceed about 800 daltons, usually does not exceed about 700 daltons and more usually does not exceed about 600 daltons. As such, low molecular weight sugars of the carbohydrate component are generally mono-, di-, and trisaccharides. Specific disaccharides of interest include sucrose, and the like. Specific trisaccharides of interest include raffinose, acarbose, maltotriose, and the like. The low molecular weight sugar component of the carbohydrate component is typically about 10 to 70% (w/w) and usually about 30 to 60% (w/w) of the total mass of the lyophilized composition. In certain embodiments, the low molecular weight sugar portion of the carbohydrate stabilizing component is a combination of a di- and trisaccharides, where the weight ratio of the di- to trisaccharides typically ranges from about 10:1 to 1:1, usually from about 5:1 to 2:1.

In addition to the low molecular weight sugar, the carbohydrate component also includes a starch. The molecular weight of the starch typically ranges from about 10 to 200 kD, usually from about 50 to 100 kD, where the starch may be linear or branched. Of interest as particular polysacharides are: dextran, glucugen, amylose, amylopectin, and the like. The amount of polysaccharide (e.g., starch) in the carbohydrate component typically ranges from about 10 to 60, usually from about 20 to 60% (w/w) of the total lyophilized composition.

In addition to the polymerase and carbohydrate components of the subject lyophilized compositions, the subject lyophilized compositions may also include a number of additional components.

In many embodiments, the lyophilized compositions include deoxyribonucleoside triphosphates (dNTPs). Usually the reaction mixture will comprise four different types of dNTPs corresponding to the four naturally occurring bases, i.e. dATP, dTTP, dCTP and dGTP. Each of the dNTPs is typically present in an amount sufficient to provide the requisite monomers during the synthesis reaction that employs the composition, where this amount typically ranges from about 0.01 to 2%, usually from about 0.05 to 1.0% and more usually from about 0.01 to 0.5% w/w of the total lyophilized composition.

The lyophilized composition further includes in many embodiments a buffer component which includes a source of monovalent ions, a source of divalent cations and a buffering agent. Any convenient source of monovalent ions, such as KCl, K-acetate, NH₄-acetate, K-glutamate, NH₄Cl, ammonium sulfate, and the like may be employed, where the amount of monovalent ion source present in the buffer component will typically be present in an amount sufficient to provide for a conductivity in a range from about 500 to 20,000, usually from about 1000 to 10,000, and more usually from about 3,000 to 6,000 micro-ohms during use. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer component is sufficient to provide for concentration in the reaction mixture prepared from the lyophilized composition (upon addition of 50 μl water) that ranges from 0.5 to 10 mM, but preferably ranges from about 2 to 5 mM. Representative buffering agents or salts that may be present in the buffer include Tris, Tricine, HEPES, MOPS and the like, where the amount of buffering agent is one that provides for a range of from about 5 to 150 mM, usually from about 10 to 100 mM, and more usually from about 20 to 50 mM in the composition prepared from the lyophilized composition upon combination with_water, where in certain preferred embodiments the buffering agent will be present in an amount sufficient to provide a pH ranging from about 6.0 to 9.5. Other agents which may be present in the buffer medium include chelating agents, such as EDTA, EGTA and the like and non-ionic detergents, such as Tween 20, Triton X100, NP40, and the like.

One component of interest that is included in many preferred embodiments of the enzyme composition is temperature sensitive polymerase inactivation means which, upon reaching a certain temperature, loses its ability to inactivate or block polymerase activity, e.g., a water-soluble temperature sensitive inhibitor of the thermostable DNA polymerase or a chemical modification to the polymerase which is inactivated upon reaching a certain temperature. Inhibitors of interest are those that bind to and inactivate the polymerase at temperature T₁ which is generally below about 85° C. For most practical purposes, T₁ is below about 55° C. Advantageously, however, the water-soluble temperature sensitive inhibitor dissociates from the DNA polymerase and becomes ineffective to inactivate the DNA polymerase at temperature T₂ which is generally above about 40° C. Preferably, T₂ is at least 5° C. above T₁. In many embodiments, T₁ is generally from about 40° C. to about 55° C. and T₂ is generally from about 75° to about 95° C. The inhibitor can be any biological or chemical molecule which will complex with the thermostable DNA polymerase to effect the noted temperature-dependent responses in the polymerase. Generally, the combined molecule (or complex) of DNA polymerase and temperature sensitive inhibitor is water-soluble. The inhibitor can be DNA polymerase-binding proteins which bind and release the DNA polymerase in response to temperature. Particularly useful inhibitors are antibodies (monoclonal or polyclonal) specific to the DNA polymerase which have the noted binding and releasing properties. The term “antibodies” includes the biological molecules one skilled in the art would normally understand that term to include, but in addition, it includes genetically prepared equivalents thereof, and chemically or genetically prepared fragments of antibodies (such as Fab fragments). The antibodies (and fragments thereof), can be used singly or in mixtures in the practice of this invention. Of particular interest in many embodiments are monoclonal antibodies. The monoclonal antibodies generally have an affinity for the thermostable DNA polymerase as defined by having an association constant of at least about 1×10⁷ molar⁻¹. Preferably, the antibody is of either the IgG or IgM class. Most preferably, it is of the IgG class. Specific monoclonal antibodies of interest include the mouse monoclonal antibodies TP1, TP2, TP3, TP4, TP5, TP6, TP7, TP8, TP9, and TP14, where these and other inhibitors of interest are further disclosed in U.S. Pat. No. 5,338,671, the disclosure of which is herein incorporated by reference.

In certain embodiments, the subject compositions may further include template nucleic acid, e.g., template DNA. Representative template nucleic acids are further described below. When present, the amount of template nucleic acid that is included in the composition ranges from about 0.000005 to 0.01, usually from about 0.00005 to 0.005. Also present in the composition in certain embodiments is one or more nucleic acid primers, e.g., a collection of gene specific primers, as described in greater detail below.

In preparing the multiwell devices having the subject lyophilized compositions present in their wells, an aqueous formulation of the various constituents of the lyophilized composition, e.g., the polymerases, the carbohydrates, the buffer component, antibody etc, is first prepared. This aqueous composition can be prepared using any convenient protocol, where the various components may be combined in any convenient or desired order, e.g., sequentially or simultaneously. A representative protocol for preparing this precursor aqueous composition is provided in the Experimental Section, infra.

The desired amount of the aqueous formulation is then placed into each well of the device in which the composition is desired. In many embodiments, the amount of aqueous composition placed, e.g., aliquoted, into each well ranges from about 1 to 100 μl, usually from about 5 to 50 μl. The resultant multi-well device having aqueous composition filled wells is then lyophilized using any convenient freeze-dry protocol. Typically, the temperature of the device is rapidly reduced, e.g., to flash freeze the device, e.g., through contact with liquid nitrogen etc. The resultant flash frozen device is then lyophilized using standard protocol, resulting in removal of substantially all free water from the composition present in each well of the device. As substantially all free water is removed from the compositions, the amount of water present in each of the compositions following the lyophilization step is typically less than about 0.1%, usually less than about 0.01% and more usually less than about 0.001% w/w, such that the compositions are correctly characterized as freeze dried compositions.

The subject high-throughput devices are further characterized in that the wells are sealed such that the lyophilized composition of each well does not come into contact with the ambient environment of the device prior to use and breaking of the seal. Any convenient sealing means may be present in the devices, so long as the sealing means and procedure for using the same does not adversely impact the nature of the lyophilized composition. The sealing means should be substantially, if not completely, water vapor proof such that water vapor cannot pass from the outside environment through the seal into the well. Because of the sealing means, a desiccant material is not present in the wells and is not required to maintain the activity of the lyophilized composition, which represents an important feature of the subject devices.

A representative sealing means of interest is a heat seal, as further described in the experimental section infra.

### METHODS

The subject devices find use in a variety of different applications, where such applications include: chain extension reactions, e.g., target generation in differential expression analyses, the polymerase chain reaction (PCR) and protocols based thereon, nucleic acid sequencing, e.g., cycle sequencing, DNA labeling, primer directed mutagenesis, and the like, where a common feature of all of these applications is template driven polymerase production of a nucleic acid. Therefore, the subject devices find use in methods of template driven or template dependent nucleic acid production.

The subject devices are particularly suited for use in the polymerase chain reaction, and applications based thereon. The polymerase chain reaction (PCR) in which a nucleic acid primer extension product is enzymatically produced from template DNA is well known in the art, being described in U.S. Pat. Nos. 4,683,202; 4,683,195; 4,800,159; 4,965,188 and 5,512,462, the disclosures of which are herein incorporated by reference.

In practicing the subject methods, an aqueous reaction mixture is first prepared by combining the lyophilized polymerase composition with template nucleic acid and primer. This aqueous reaction mixture is conveniently prepared by adding an appropriate amount of water and the other requisite components, e.g., template, primer etc., to the one or more wells of device containing the lyophilized composition. Where the device is sealed, this step further includes breaking the seal. A feature of the subject methods is that this step does not require handling and measurement of the lyophilized composition. Instead, all that is required is placement of the appropriate amount of water, template and primer into the well that contains the lyophilized composition. The amount of water that is added to the well typically ranges from about 1 to 200 μl, usually from about 5 to 100 μl. The water may be added using any convenient protocol, e.g., by pipette, automatic dispensation, etc.

The nucleic acid that serves as template may be single-stranded or double-stranded, where the nucleic acid is typically deoxyribonucleic acid (DNA), where when the nucleic acid is single stranded, it will typically be converted to double stranded nucleic acid using one of a variety of methods known in the art. The length of the template nucleic acid may be as short as 50 bp, but usually be at least about 100 bp long, and more usually at least about 150 bp long, and may be as long as 10,000 bp or longer, but will usually not exceed 50,000 bp in length, and more usually will not exceed 20,000 bp in length. The nucleic acid may be free in solution, flanked at one or both ends with non-template nucleic acid, present in a vector, e.g., plasmid and the like, with the only criteria being that the nucleic acid be available for participation in the primer extension reaction. The template nucleic acid may be derived from a variety of different sources, depending on the application for which the PCR is being performed, where such sources include viruses; prokaryotes, e.g. bacteria, archaea and cyanobacteria; and eukaryotes, e.g. members of the kingdom protista, such as flagellates, amoebas and their relatives, amoeboid parasites, ciliates and the like; members of the kingdom fungi, such as slime molds, acellular slime molds, cellular slime molds, water molds, true molds, conjugating fungi, sac fungi, club fungi, imperfect fungi and the like; plants, such as algae, mosses, liverworts, hornworts, club mosses, horsetails, ferns, gymnosperms and flowering plants, both monocots and dicots; and animals, including sponges, members of the phylum cnidaria, e.g. jelly fish, corals and the like, combjellies, worms, rotifers, roundworms, annelids, mulloses, arthropods, echinoderms, acorn worms, and vertebrates, including reptiles, fishes, birds, snakes, and mammals, e.g. rodents, primates, including humans, and the like. The nucleic acid may be used directly from its naturally occurring source and/or preprocessed in a number of different ways, as is known in the art. In some embodiments, the nucleic acid may be from a synthetic source.

The amount of template nucleic acid that is combined with the other reagents in the well to produce the reaction mixture in the will range from about 1 molecule to 1 pmol, usually from about 50 molecules to 0.1 pmol, and more usually from about 0.01 amol to 100 fmol.

The oligonucleotide primers employed in the subject methods are of sufficient length to provide for hybridization to complementary template DNA under annealing conditions (described in greater detail below) but will be of insufficient length to form stable hybrids with non-complementary template DNA. The primers will generally be at least 10 bp in length, usually at least 15 bp in length and more usually at least 16 bp in length and may be as long as 30 bp in length or longer, where the length of the primers will generally range from 18 to 50 bp in length, usually from about 20 to 35 bp in length. The template DNA may be contacted with a single primer or a set of two primers, depending on whether linear or exponential amplification of the template DNA is desired. Where a single primer is employed, the primer will typically be complementary to one of the 3′ ends of the template DNA and when two primers are employed, the primers will typically be complementary to the two 3′ ends of the double stranded template DNA.

Following preparation of the reaction mixture, the reaction mixture is subjected to one or more template driven chain extension reactions. For PCR applications, the reaction mixture is subjected to a plurality of reaction cycles, where each reaction cycle comprises: (1) a denaturation step, (2) an annealing step, and (3) a polymerization step. The number of reaction cycles will vary depending on the application being performed, but will usually be at least 15, more usually at least 20 and may be as high as 60 or higher, where the number of different cycles will typically range from about 20 to 40. For methods where more than about 25, usually more than about 30 cycles are performed, it may be convenient or desirable to introduce additional polymerase into the reaction mixture such that conditions suitable for enzymatic primer extension are maintained.

The denaturation step comprises heating the reaction mixture to an elevated temperature and maintaining the mixture at the elevated temperature for a period of time sufficient for any double stranded or hybridized nucleic acid present in the reaction mixture to dissociate. For denaturation, the temperature of the reaction mixture will usually be raised to, and maintained at, a temperature ranging from about 85 to 100, usually from about 90 to 98 and more usually from about 93 to 96° C. for a period of time ranging from about 3 to 120 sec, usually from about 5 to 60 sec.

Following denaturation, the reaction mixture will be subjected to conditions sufficient for primer annealing to template DNA present in the mixture. The temperature to which the reaction mixture is lowered to achieve these conditions will usually be chosen to provide optimal efficiency and specificity, and will generally range from about 50 to 75, usually from about 55 to 70° C. Annealing conditions will be maintained for a period of time ranging from about 15 sec to 60 sec.

Following annealing of primer to template DNA or during annealing of primer to template DNA, the reaction mixture will be subjected to conditions sufficient to provide for polymerization of nucleotides to the primer ends in manner such that the primer is extended in a 5′ to 3′ direction using the DNA to which it is hybridized as a template, i.e. conditions sufficient for enzymatic production of primer extension product. To achieve polymerization conditions, the temperature of the reaction mixture will typically be raised to or maintained at a temperature ranging from about 65 to 75, usually from about 67 to 73° C. and maintained for a period of time ranging from about 15 sec to 20 min, usually from about 30 sec to 5 min.

The above cycles of denaturation, annealing and polymerization may be performed using an automated device, typically known as a thermal cycler. Representative thermal cyclers are described in U.S. Pat. Nos. 5,612,473; 5,602,756; 5,538,871; and 5,475,610, the disclosures of which are herein incorporated by reference.

The subject template driven nucleic acid production methods find use in any application where the production of enzymatically produced primer extension product from template DNA is desired, such as in the generation of specific sequences of cloned double-stranded DNA for use as probes, the generation of probes specific for uncloned genes by selective amplification of particular segments of cDNA or genomic DNA, the generation of libraries of cDNA from small amounts of mRNA, the generation of large amounts of DNA for sequencing, the analysis of mutations, generation of DNA fragments for gene expression, chromosome crawling, and the like. Thus, the subject methods of PCR find use in diagnosis, such as of genetic disorders and identification of pathogens; in genetic identification of forensic samples, in the analysis of mutations, and the like. See PCR, Essential Techniques, (ed J. F. Burke, John Wiley & Sons) (1996).

The subject methods are characterized in that the lyophilized polymerase mixture provides for substantially the same results in terms of template driven nucleic acid production as are observed with a comparable freshly prepared polymerase mixture, i.e., a mixture that has not be lyophilized. Substantially the same results are considered to be achieved where at least one of nucleic acid length, efficiency and fidelity are substantially the same, where one of these parameters is considered be substantially the same if it does not vary by more than about 10%, and preferably by not more than about 5%. For example, the difference in nucleic acid length achieved with the subject lyophilized compositions as compared to a freshly prepared composition, i.e., the same composition prior to lyophilization, does not vary by more than about 10%, and usually does not vary by than about 5%.

### KITS

As summarized above, also provided are kits for use in practicing the subject methods. The kits at least include the subject high throughput devices, as described above. The kits may also include a number of optional components that find use in the subject methods. Optional components of interest include

The kits may further include one or more nucleic acids, where the nucleic acids will generally be oligonucleotides that find use in the subject methods, described in greater detail above. As such, nucleic acids that may be present include random primers and PCR primers. Also present may be one or more sets of PCR primers, where such primers may be control primers etc. In certain embodiments, the primers may be a set of gene specific primers, as described in U.S. Pat. No. 5,994,076, the disclosure of which is herein incorporated by reference.

The subject kits may also include template nucleic acid, e.g., template DNA as described above.

Other optional components that may be included in the subject kits include: one or more control sets of template nucleic acid and appropriate primers that specifically hybridize to the template nucleic acid, etc.

Finally, in many embodiments of the subject kits, the kits will further include instructions for practicing the subject methods or means for obtaining the same (e.g., a website URL directing the user to a webpage which provides the instructions), where these instructions are typically printed on a substrate, which substrate may be one or more of: a package insert, the packaging, reagent containers and the like.

The following examples are offered by way of illustration and not by way of limitation.

### EXPERIMENTAL

I. Manufacture of High Throughput Device

A. Preparation of PCR Composition:

Produce a mixture having the following components:

| | | |
|---|---|---|
| 3.2% | | Dextran (Sigma Cat. # D9751) |
| 4.8% | | Sucrose (Sigma Cat. # S03 89) |
| 1.6% | | Raffinose (Sigma Cat. #R0514) |
| 80 | μM | K-Tricine (pH 8.7) |
| 30 | μM | K-acetate |
| 7 | μM | Mg-acetate |
| 7.5 | μg/μl | BSA |
| 0.01% | | Tween20 |
| 0.01% | | NP40 |
| 400 | μM | dATP |
| 400 | μM | dGTP |
| 400 | μM | dCTP |
| 400 | μM | dTTP |
| 1 | μ/μl | Advantaq polymerase (Clontech) |
| 0.044 | μg/μl | TaqStart Antibody (Clontech) |
| 0.00035 | μ/μl | Deep Vent polymerase (New England Biolabs) |

and aliquot 25 μl of the resultant composition into the PCR wells of a multi-well polypropylene PCR plate, either manually or robotically.

B. Lyophilization

Freeze the PCR plate, e.g. by contacting with N₂ and lyophilize.

C. Heat Seal

Following Lyophilization the plates are heat sealed as follows. First a laminate heat seal film (foil) of aluminum/polypropylene (ABgene, UK) is placed across the openings of the wells in the plate. Next, the foil is pressed with a hot plate ('80° C.) for a approximately 5 sec. This process results in a heat sealed multiwell PCR plate that includes a lyophilized polymerase composition in each well.

II. Testing of High Throughput Device

A. 5′,3′ RACE PCR Assay

The ability of the high throughput device to be used in a 5′,3′ RACE PCR assay (see e.g., SMART™ RACE cDNA Amplification Kit, Clontech Laboratories, Palo Alto, Calif.) was evaluated as follows. Advantage 2 (Clontech Laboratories Inc., Palo Alto, Calif.) control samples were prepared according to the manufacturer's protocol. The lyophilized samples of the high throughput device as prepared in Example I were reconstituted with a total of 50 μl of Milli-Q water containing the proper amount of template and primers as per application.

1) RACE-PCR

Lane 1—Sprint sample, 5′ RACE;

Lane 2—Sprint sample, 3′ RACE;

Lane 3—Advantage 2 control, 5′ RACE;

Lane 4—Advantage 2 control, 3′ RACE

In each PCR, 5 μl pf PCR control cDNA (from Marathon cDNA Amplification kit, K1802-1, Clontech Laboratories, Palo Alto, Calif.) was used as a template. For 5′ RACE, the 5′ RACE-primer and AP1 primer (from the same kit) were used. Likewise, for 3′ RACE, 3′ RACE-TFR and AP1 primers were used.

PCR was as follows: 94° C./5 sec and 72° C./3 min for 5 cycles, followed by 94° C./5 sec, 70° C./10 sec and 72° C./3 min for 5 cycles, followed by 94° C./5 sec, 68° C./10 sec and 72° C./3 min for 27 cycles. 5 μl of the PCR product were electrophoresed on 1.2% agarose in TAE gel. In 5′ RACE, a 2.6 kb band (amplified fragment from the TFR gene) was observed, and in the 3′ RACE reaction, a major band at 2.9 kb was observed (again, the TFR gene—a minor band is also seen below).

Importantly, there was no observable difference in the results obtained using the fresh Advantage 2 mixture as compared to those results obtained using the high throughput device of Example I.

B. LD-PCR Assay

The ability of the high throughput device to be used in LD-PCR (see e.g., Product Catalog: Marathon™ cDNA Amplification Kit, Clontech Laboratories, Palo Alto, Calif.) was evaluated as follows. Advantage 2 prepared as described above was compared to the high throughput device of Example I.

Lane 1,2—Advantage 2 controls

Lane 3,4—samples run in the subject high throughput devices

In each PCR, 1 μl pf EMBL3/1 GEM11 lysate was used, containing an 18.7 kb insert (from the Advantage Genomic PCR kit, K1906-1, Clontech Laboratories, Palo Alto Calif.) as the template along with the LD-insert screening primer for the same vector.

PCR was as follows: 95C/1 min, followed by 95C/15 sec and 70C/12 min for 20 cycles, followed by 70C/12 min. 5 μl of the PCR product was electrophoresed on 1.2% agarose in TAE gel. A single band of 18.7 kb was observed in all lanes. No visible difference could be detected between lanes, indicating that the subject high throughput device worked just as well as, i.e., substantially the same as, the freshly prepared polymerase mix (i.e., Advantage 2), in the LD-PCR.

It is evident from the above results and discussion that the subject invention provides a convenient and useful high throughput device for performing template dependent primer extension reactions, such as PCR. Advantages provided by the subject invention include pre-measured reagents that require no handling beyond the addition of water and a format that is readily adaptable to high throughput applications. The subject advantages are provided without any comprise in terms of efficiency and/or fidelity in the reaction. For example, the long distance PCR embodiments of the subject devices provide for substantially the same results in terms of efficiency and fidelity of reaction product as is observed using freshly prepared long distance PCR reaction mixtures. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A substrate having at least one well filled with a lyophilized nucleic acid polymerase composition comprising: (a) at least one polymerase; and (b) a carbohydrate stabilizing composition comprising: (i) at least one low molecular weight sugar; and (ii) at least one polysaccharide.

2. The substrate according to claim 1, wherein said lyophilized polymerase composition comprises a nuclease deficient polymerase and a proof-reading polymerase.

3. The substrate according to claim 2, wherein said lyophilized composition further comprises at least one buffer component, nucleotides and an antibody specific for said nuclease deficient polymerase.

4. The substrate according to claim 1, wherein said carbohydrate stabilizing composition includes: a disaccharide, a trisaccharide and a starch.

5. The substrate according to claim 3, wherein said carbohydrate stabilizing composition includes: a disaccharide, a trisaccharide and a starch.

6. The substrate according to claim 5, wherein said disaccharide is sucrose, said trisaccharide is raffinose and said starch is dextran.

7. The substrate according to claim 1, wherein said substrate is sealed.

8. A lyophilized polymerase composition comprising:
(a) a polymerase component that is made up of first polymerase that lacks nuclease activity and a second proofreading polymerase; and
(b) a carbohydrate stabilizing composition comprising:
(i) at lease one low molecular weight sugar; and
(ii) at least one starch.

9. The composition according to claim 8, wherein said composition further comprises at least one buffer component and nucleotides.

10. The composition according to claim 8, wherein said composition further comprises an antibody specific for said polymerase.

11. The composition according to claim 9, wherein said composition further comprises an antibody specific for said nuclease deficient polymerase.

12. The composition according to claim 8, wherein said carbohydrate stabilizing composition includes: a disaccharide, a trisaccharide and a starch.

13. The composition according to claim 11, wherein said carbohydrate stabilizing composition includes: a disaccharide, a trisaccharide and a starch.

14. The composition according to claim 13, wherein said disaccharide is sucrose, said trisaccharide is raffinose and said starch is dextran.

15. The composition according to claim 14, wherein said composition is present in at least one well of a multi-well substrate.

16. A method of producing a nucleic acid using a template dependent nucleic acid polymerase reaction, said method comprising:
(A) providing a substrate having at least one well filled with a lyophilized polymerase composition comprising:
(1) at least one polymerase; and
(2) a carbohydrate stabilizing composition comprising:
(i) at lease one low molecular weight sugar; and
(ii) at least one starch; and
(B) contacting said lyophilized composition in said at least one well with water and a template under template dependent polymerase reaction conditions to produce said nucleic acid.

17. The method according to claim 16, wherein said lyophilized polymerase composition comprises a nuclease deficient polymerase and a proof-reading polymerase.

18. The method according to claim 17, wherein said lyophilized composition further comprises at least one buffer component, nucleotides and an antibody specific for said nuclease deficient polymerase.

19. The method according to claim 17, wherein said carbohydrate stabilizing composition includes: a disaccharide, a trisaccharide and a starch.

20. The method according to claim 19, wherein said carbohydrate stabilizing composition includes: a disaccharide, a trisaccharide and a starch.

21. The method according to claim 20, wherein said disaccharide is sucrose, said trisaccharide is raffinose and said starch is dextran.

22. The method according to claim 16, wherein said substrate is sealed and said method further comprises breaking said seal.

23. A kit for use in a method of producing a nucleic acid using a template dependent nucleic acid polymerase reaction, said kit comprising:
a sealed multi-well substrate having at least one well filled with a lyophilized polymerase composition comprising: (a) at least one polymerase; and (b) a carbohydrate stabilizing composition comprising: (i) at lease one low molecular weight sugar; and (ii) at least one starch.

24. The kit according to claim 23, wherein said kit further comprises at least one nucleic acid primer.

25. The kit according to claim 23, wherein said kit further comprises water.

26. The kit according to claim 23, wherein said kit further comprises a nucleic acid template.

27. The kit according to claim 23, wherein said kit further comprises instructions for producing a nucleic acid using a template dependent polymerase reaction with the components of said kit.
